(19) United States
(12) Patent Application Publication
(10) Pub. No.: US 2025/0059396 A1
(43) Pub. Date: Feb. 20, 2025

(54) LEAD OR CHALK FOR WRITING, PAINTING AND/OR COSMETIC PURPOSES, PENCIL WITH A LEAD AND METHOD FOR PRODUCING A LEAD OR CHALK

(71) Applicant: Faber-Castell AG, Stein (DE)

(72) Inventors: Baukis HEULER, Nurnberg (DE); Christian POLLMANN, Furth (DE); Gerhard LUGERT, Nurnberg (DE)

(21) Appl. No.: 18/782,081

(22) Filed: Jul. 24, 2024

(30) Foreign Application Priority Data
Jul. 24, 2023 (EP) 23187376.1

(51) Int. Cl. C09D 13/00; C09D 1/00; C09D 7/63; C09D 191/00; C09D 191/06

(52) U.S. Cl. CPC C09D 13/00; C09D 1/00; C09D 7/63; C09D 191/005; C09D 191/06

(57) ABSTRACT

The invention relates to a biodegradable and/or compostable core or crayon for writing, drawing, and/or cosmetic purposes containing at least one fatty acid ester, at least one metal soap and/or soap, and at least one colorant, wherein the at least one fatty acid ester and the at least one metal soap and/or soap are included in a total proportion of 33 to 90 wt. %.

The invention furthermore relates to a pencil comprising such a core and to a method for producing such a core or crayon.

The invention relates to a core or crayon for writing, drawing, and/or cosmetic purposes, and to a pencil having such a core. The invention furthermore relates to a method for producing a core or crayon.

Thermoplastically producible waterproof cores using polystyrene, polyvinylbutyral, or other thermoplastics as binders are known. Although such cores have sufficient strength, they are not optimal with respect to their coating behavior because relatively high forces are required to achieve sufficient opacity when creating a coating. EP 2 520 442 B1 describes, for example, colored pencils or crayons, the composition of which is based on fats and waxes, in addition to fillers and colorants, wherein said fats and waxes are contained in a proportion of a maximum of 30 wt. %. Furthermore, there are thermoplastically producible, water-soluble, water-colorable colored pencil cores that contain sugar and are described, for example, in EP 1 552 958 B1 and EP 3 272 819 B1. These cores have good mechanical strength and can be produced with diameters between 2.8 and 6 mm. It is also possible to produce crayons having diameters greater than 6 mm.

It is a drawback of the aforesaid cores that, in particular with waterproof cores, plastics are used as binders and/or wax components are used and contain petrochemical polymers that are at least in part non-biodegradable. In addition, with the aforesaid cores it is not possible to produce cores that can be applied very softly.

It is therefore the object of the invention to provide a core or crayon for writing, drawing, and/or cosmetic purposes that can be adjusted to be both water-soluble and waterproof and which is improved in terms of the binder it contains. It is furthermore the object of the invention to provide a pencil having a core and a method for producing a core or crayon that permits cost-effective and efficient production of cores or crayons.

The object is attained with a biodegradable and/or compostable core or crayon for writing, drawing, and/or cosmetic purposes, in particular a colored core or crayon having the features according to claim 1. According to the latter, at least one fatty acid ester, at least one metal soap, that is, a metal salt of a fatty, resin, or napthenic acid, and/or soap, that is, a water-soluble fatty acid salt of the sodium and potassium salts, and at least one colorant are contained in the core or crayon. According to the invention, the at least one fatty acid ester and the at least one metal soap and/or soap are contained in the core or crayon in a proportion of 33 to 90 wt. %, in particular in a proportion of at least 35 wt. %, and/or in a proportion of a maximum of 80 wt. %.

In other words, fatty acid esters and metal salts are combined in the core or crayon. The aforesaid content range relates to the total proportion of the fatty acid esters, metal soap(s), and/or soap(s) contained in the core or crayon relative to the entire core or crayon.

The at least one fatty acid ester is contained in a proportion of 2 to 30 wt. %, the at least one metal soap and/or soap in a proportion of 10 to 70 wt. %.

In this case biodegradable and/or compostable core or crayon shall be understood to be a core or crayon that does not contain a binder that must not have degraded more than 90 percent to water, CO2, and biomass within a specified time under defined temperature, oxygen, and moisture conditions in the presence of microorganisms or fungi. In this case, at least one fatty acid ester, at least one metal soap and/or soap is contained as binder. For the sake of simplicity, the following refers primarily to a core, but the explanations also apply to a crayon.

The core or crayon is produced from a core base substance or core starting material containing the aforesaid components or possibly further starting materials that is thermoplastically processed and extruded in order to melt and bind the starting materials at an elevated temperature. Likewise, the core can be produced from starting materials that react chemically with one another during the thermoplastic processing in order to obtain a core substance or core containing fatty acid esters and metal soaps and/or soaps. In this case, saponification thus occurs during the thermoplastic processing. Any water added during the production of the core to promote mixing of the starting materials or any water occurring during the chemical reaction is then removed again, in particular completely removed, so that the proportion of free water is reduced to a maximum of 0.5 wt. %, in particular 0 wt. %. The core substance and the core itself are thus anhydrous and contain at most 0.5 wt. % water. Core or crayon strands are then formed by means of extrusion and the core or crayon strands are cut to length to form cores or crayons. The core or crayon is already finished once the core substance has cooled; no complex removal of water or solvents is required.

The use of fatty acids or fatty acid esters and metal salts of fatty acids in extruded colored pencil cores is well known. To date, the fatty acids have usually been added to water-proof cores as a low-melting-point wax to improve core release when writing or drawing, small amounts (≤12%) of metal salts being added as gelling agents. In water-soluble extruded cores, the starch acts as a thickener, fatty acids act as low-melting-point wax for better release, and soap is added here at about 50% of the binder to improve water solubility.

After numerous tests, it was surprisingly found that metal soaps and/or soaps in larger quantities can function as binders in cores and crayons and in addition create a new approach for producing both waterproof and water-soluble cores in a thermoplastic method. The cores have good mechanical strength and demonstrate good application behavior. In addition, they can be produced cost-effectively and efficiently in a thermoplastic process. Improved color release with low pressure and better covering over ability is obtained due to the contained metal soap and/or soap or the saponification during production.

In particular, at least one fatty acid ester and/or at least one metal soap(s) and/or soap(s) based on natural fats, waxes, and/or oils are included that comprise a mixture of different carboxylic acids that differ in chain length, number, distribution, and direction of double bonds. Natural fatty acids are preferably used for producing biodegradable and/or compostable bio-based cores and crayons. The longer the chain of the fatty acids, the higher their melting point and breaking force, but the slower the saponification reaction during production and the less release there is when writing and drawing. The more double bonds the fatty acid has, the lower its melting point and the breaking force decreases. In contrast, release onto the writing or drawing surface increases. Combinations of short-chain fatty acids or fatty acids with double bonds for release and long-chain fatty acids for stability are well suited for cores, wherein a range of chain lengths C8 (from coconut oil) to C30 (from beeswax) is recommended. Where appropriate, at least one fatty acid ester and/or at least one metal soap and/or soap based on a resin acid and/or a napthenic acid and/or a polar wax may be included, wherein their entire proportion is limited to a maximum of 5 wt. % in order to obtain the biodegradability of the core.

Preferably at least one fatty acid ester and/or at least one metal soap and/or soap based on a carboxylic acid, in particular a fatty acid such as stearic acid, palmitic acid, oleic acid, lauric acid, myristic acid, capric acid, palmitoleic acid, linoleic acid, rice bran wax, sunflower wax, is included.

Moreover, the at least one metal soap and/or soap is based in particular on metal ions of monovalent salts of alkali metals, in particular lithium, sodium, and/or potassium, and/or on metal ions of divalent alkaline earth metals, in particular magnesium, calcium, and barium, and or on metal ions of metals, in particular aluminum and zinc is included. A combination of different metal ions is also possible, as is the use of further anionic or cationic surfactants, such as, e.g., sodium cocoyl isethionate.

Cores or crayons can preferably contain a soap based on a fatty acid, in particular a fatty acid up to a maximum of C16 and/or a mixture of longer-chain fatty acids than C18, such as, e.g., stearic acid, and fatty acids up to a maximum of C16, and based on sodium and/or potassium as metal ion. The core or crayon is water-soluble due to fatty acids up to a maximum of C16. An improvement in water solubility can also be achieved by increasing the sodium or potassium ions or the proportion of soap compared to the fatty acid.

In accordance with one further advantageous embodiment, the core or crayon contains a metal soap based on the cations lithium, magnesium, calcium, barium, aluminum, and zinc as metal ion and/or based on sodium and/or potassium as metal ion in combination with a longer-chain fatty acid than C18, e.g. stearic acid. The core or crayon is waterproof because of this. It is also possible to use potassium or sodium ions in the waterproof formulation, but only in combination with long-chain fatty acids C18 or longer-chain fatty acids. It is also possible to use shorter-chain fatty acids, but only in a low ratio to the longer-chain fatty acids, depending on the actual chain length. Reducing the proportion of cations also leads to a reduction in the water solubility of the cores and thus promotes waterproofing.

The metal salts of fatty acids required for production can either be used as soap for core production. It is also possible, however, to carry out saponification of a fatty acid with a metal hydroxide or metal salt, e.g., alkaline metal, alkaline earth metal, or metal carbonates or phosphates, in the production process.

In accordance with one advantageous embodiment, a mass ratio of the fatty acid(s) to metal soap(s) and/or soap(s) in the core or crayon is between 1:1 and 1:10. In other words, the proportion of fatty acid(s) to metal acid(s) and/or soap(s) is selected such that the core or crayon has an excess fat content between 10 and 50%. The fatty acids and metal soap(s) and/or soap(s) or the metal hydroxides used for saponification are thus combined such that an excess of fatty acids (superfatting)<50% is attained.

The lower the superfatting, the higher the proportion of metal ions compared to fatty acid(s) and the application becomes less smooth. On an even surface there is significantly better release, even without pressure. In addition, overpaintability is improved. Superfatting of less than 10% is not effective, since the breaking force decreases substantially. Lower superfatting leads to the melting point of the starting materials increasing during production and the melt having a higher viscosity. This permits achievement of better dimensional stability of the cores at high temperatures, that is, e.g., when cutting or trimming the core strands following extrusion, or if the product is potentially to be stored at high temperatures, e.g., in an overseas container or the like.

In one preferred refinement, the colorant is contained in the core or crayon in a proportion of 0.1 to 35 wt. %, in particular in a proportion of 0.1 to 25 wt. %.

Preferably dyes and/or organic pigments and/or inorganic pigments are included as colorants, in particular finely powdered organic and/or inorganic pigments having a particle size distribution D90<100 μm. Typical organic pigments are those from the group of phthalocyanine pigments, azo pigments, indanthrone pigments, perylene pigments, and diketopyrrolopyrrole pigments (DPP). Iron oxides, titanium dioxide, manganese violet, ultramarine blue, milori blue, as well as carbon black and other colorants are possible as inorganic pigments. Cores and crayons whose pigments have such a particle size distribution, due to the high proportion of metal soap(s) and/or soap(s) in the present core, apply with good color intensity without scratching or uneven color release, as would typically occur with coarser pigments. The particle size of the pigments can be determined using various techniques known to the person skilled in the art. For example, particle size and particle size distribution of the pigment can be determined using laser diffraction. In the present case, the Malvern Panalytical Mastersizer 3000 device was used to determine particle size and particle size distribution.

As a rule, fillers are used in classic colored pencil cores. Using the combination of fatty acids and metal soaps and/or soaps as binders, it is generally possible to produce a filler-free core. However, one or a plurality of fillers may be included in order to influence the property profile of the core or crayon, in particular the application behavior and breaking force. The addition of a filler at a maximum proportion of 75 wt. % in the core or crayon has proven advantageous. Fillers are generally natural raw materials such as inorganic minerals (kaolin, CaCO₃, clay, quartz powder, talcum, mica) or plant fibers (wood chips, cellulose). The breaking force of the core reaches a maximum depending on the filler content and diminishes again if the binder content, that is the proportion of fatty acid ester(s) and metal soaps and/or soaps, is no longer sufficient for binding the solids, that is the non-meltable substances such as fillers, pigments, and even mineral additives.

In addition, additives can be employed that on the one hand act as processing aids. In addition, by employing additives it is sometimes possible to control the water resistance or water solubility in the core composition. According to one advantageous refinement, the core or crayon contains at least one additive. The maximum proportion of additives is in particular 15 wt. %. Hydroxyls, e.g. glycerin or sugar, but also carboxylic acids, such as lactic acid, citric acid, acetic acid, and/or salts may be included as possible additives. The additives can influence breaking force, application behavior, color shade, and water solubility of the core or crayon.

Hydroxyls are classified as alcohols and polyols according to the number of hydroxyl groups they contain. A plurality of hydroxyl groups, for example in starch or cellulose, permit cross-linking of the hydroxyls and fatty acids, in particular carboxylic acids. Employment of such hydroxyls causes the breaking force and deflection of the cores to increase and application to slow somewhat. In addition to the number of hydroxyl groups, chain length is also relevant. Long-chain hydroxyls, such as, e.g. in sunflower wax (C26-C32), increase breaking force, melting point, and water resistance, while reactivity and biodegradability diminish. Application is smoother.

The use of special carboxylic acids, such as acetic acid, lactic acid, and citric acid, as an additive is also possible. For example, citric acid is particularly short-chain and thus is already suitable as rapid reactant even in small quantities of up to 0.5 wt. %. Citric acid has a plurality of acid groups and can thus be used as a cross-linking additive, so that the hardness of the core is increased. Lactic acid has both a carboxyl and hydroxyl group, so that they can esterify with one another.

Water solubility, hygroscopy, and hardness of the core or crayon can be influenced by the employment of salts.

The composition of the inventive cores or crayons can thus be given as follows:

| | |
|---|---|
| Fatty acid ester and metal soap(s) | 33 to 90 wt. % |
| and/or soap(s) | |
| Filler | 0 to 75 wt. % |
| Colorant | 0.1 to 35 wt. % |
| Additive | 0 to 15 wt. % |

The object is further attained with a pencil having the features according to claim 12, which includes a core having the features described in the foregoing. In particular the pencil has a casing made of wood or a wood substitute that encases the core. A so-called wood-plastic compound is used as a wood substitute instead of natural wood, so that, for example, the pencil, can be produced in a simple manner using co-extrusion of the core and casing.

Moreover, the object is attained using a method for producing a biodegradable and/or compostable core or a crayon having the features according to claim 13. According to the method, the starting materials required for producing the core or the substances contained in the core starting substance, that is, the at least one fatty acid ester, the at least one metal soap and/or soap or the metal hydroxides and/or metal salts reacting to form the metal soap and/or soap, and, in particular, at least one further fatty acid ester, and the at least one colorant are thermoplastically processed or mixed and/or homogenized and bonded to one another at a temperature that causes the starting materials to melt. Where necessary, further substances, such as, for example, fillers or additives are added and thermoplastically processed together with the aforesaid starting materials.

The core base substance obtained therefrom is extruded to form cores or crayon strands. The at least one fatty acid ester, the at least one metal soap and/or soap or the fatty acids and metal hydroxides reacting to form metal soap and/or soap are added such that the at least one fatty acid ester and the at least one metal soap and/or soap are contained in the core or crayon in a proportion of 33 to 90 wt. %.

The following can be provided as the basic formulation for the core or the starting materials required for producing the core or the substances contained in the core starting substance:

| | |
|---|---|
| Binder fatty acids + metal ions/ | 33 to 90 wt. % |
| metal hydroxides or metal salts of fatty acids | |
| Filler | 0 to 75 wt. % |
| Colorant | 0.1 to 35 wt. % |
| Additive | 0 to 15 wt. % |

In other words, during production a fatty acid ester and metal soaps already present as such, that is, fatty acid esters, can be used as starting materials. If the metal soaps and/or soaps are not yet present as such, at least one fatty acid ester and metal hydroxides and/or metal salts that react to form the metal soap and/or soap and possibly further fatty acid esters are added for producing the core or crayon and saponification of the fatty acid ester takes place during the production process.

The core or crayon can be produced using a wet or dry method.

Water is preferably added to the starting materials, in particular in a proportion of about 5 to 20 wt. %, and is thermoplastically processed with the at least one fatty acid, the at least one metal soap and/or soap or the fatty acids and metal hydroxides reacting to form metal soap and/or soap, and the at least one colorant, wherein the proportion of the free water is reduced during mixing to at most 0.5 wt. %, preferably 0 wt. % (wet method). Due to the reduction in the water content, the extruded or molded core base substance has a low moisture content, so that the finished core is already available as soon as the core base substance has cooled. There is no need for complex removal of water or other solvents.

The at least one fatty acid ester, the at least one metal soap and/or soap or metal hydroxides and/or metal salts reacting to form metal soap and/or soap, and in particular the at least one further fatty acid ester, and the at least one colorant are preferably thermoplastically processed at a temperature of at least 50° C.

Fatty acid esters and/or metal soaps and/or soaps employed are preferably those based on natural fats, waxes, and/or oils.

Preferably a fatty acid ester and/or a metal soap or a soap based on a carboxylic acid is used, in particular a fatty acid such as stearic acid, palmitic acid, oleic acid, lauric acid, myristic acid, capric acid, palmitoleic acid, linoleic acid, rice bran wax, sunflower wax, and/or a metal soap or soap based on metal ions of monovalent salts of the alkaline metals, in particular lithium, sodium, and/or potassium, and/or based on metal ions of divalent alkaline earth metals, in particular magnesium, calcium, and barium, and/or based on metal ions of metals, in particular aluminum and zinc.

In particular lithium, sodium, magnesium, calcium, barium, aluminum, and zinc hydroxides are employed as metal hydroxides.

For producing water soluble cores and crayons, a soap based on a fatty acid, in particular a fatty acid to maximum C16 and/or a mixture of longer chain fatty acids than C18 and fatty acids to maximum C16, and based on sodium and/or potassium as metal ion, or a fatty acid, in particular a fatty acid to maximum C16 and/or a mixture of longer chain fatty acids than C18 and short chain fatty acids to maximum C16 and sodium hydroxides and/or potassium hydroxides and/or potassium carbonates are preferably added.

In order to produce waterproof cores or crayons, preferably a metal soap based on lithium, magnesium, calcium, barium, aluminum, and/or zinc as metal ion and/or based on sodium and/or potassium as metal ion are preferably added in combination with a longer chain fatty acid than C18, or lithium, magnesium, calcium, barium, aluminum, or zinc hydroxides and/or sodium hydroxides and/or potassium hydroxides in combination with a longer chain fatty acid than C18.

The fatty acid(s) and metal soap(s) and/or soap(s) and/or metal hydroxides are added in particular such that the ratio of fatty acid(s) to metal soap(s) and/or soap(s) in the core or crayon is between 1:10 to 10:10 in order to achieve superfatting of between 10 and 50%.

Preferably the colorant is added such that the latter is contained in the core in a proportion of 0.1 to 35 wt. %, in particular a proportion of 0.1 to 25 wt. %. Dyes and/or organic pigments and/or inorganic pigments can be added as colorants, in particular organic and/or inorganic pigments having a particle size distribution of D90<100 μm.

Furthermore, at least one filler, preferably calcium carbonate, kaolin, talc, pumice powder, quartz powder, mica, white pigments, and/or plant fibers can be added, in particular such that the aforesaid is contained in the core or crayon in a maximum proportion of 75 wt. %.

According to one advantageous refinement, at least one additive, preferably selected from the group of hydroxyls and/or carboxylic acids and/or salts, is added, in particular such that the aforesaid is contained in a maximum proportion of 15 wt. %.

The following provides seven exemplary formulations for a biodegradable and/or compostable core or crayon. Such cores can be produced for example using the so-called “wet method”. In this case, dry raw materials or starting materials are added to a high-speed mixer or kneader and homogenized during the mixing process. Then water is added, in particular in a proportion of 5 to 20 wt. %. The water-soluble components are dissolved using subsequent heating to temperatures greater than 100° C., preferably at reduced speed. Then further drying of the mass can be carried out by opening the mixer or kneader. Saponification of the added metal hydroxides and fatty acids takes place during the method.

Alternatively, it is possible to produce the cores using a so-called “dry method”. This is recommended in particular when the metal soaps and/or soaps, that is, the fatty acid salts, are already present and moreover only meltable materials are used as additives, such as, e.g. sorbitol or dextrin. To this end, all raw materials or starting materials are added to a high-speed mixer or extruder. The heating phase takes place up to the melting point at temperatures greater than 100° using high speeds. Then the residual moisture of the materials escapes in a “dry phase” of the mixing process at a temperature below the melting temperature.

In both the wet and dry methods, the granulate obtained is then cooled and extruded into core or crayon strands, for example on one or preferably two screw extruders or piston extruders. The core or crayon strands are then cut to length.

### EXAMPLE 1: RED WATER-SOLUBLE CORE USING WET METHOD (SAPONIFICATION DURING MIXING)

Starting formulation:

| | | |
|---|---|---|
| Stearin | 24 | wt. % |
| Rapeseed oil | 12 | wt. % |
| Potassium hydroxide | 4 | wt. % |
| Kaolin | 55 | wt. % |
| Lithopone DS 30 | 3 | wt. % |
| Pigment 1 (Pigment red 254 5 CI 56110) | 1 | wt. % |
| Pigment 2 (Pigment red 122) | 1 | wt. % |

A core or crayon or a pencil having a core with a composition as in Example 1 is produced using the wet method. In addition, 15 wt. % water is added and completely evaporates by the end of the mixing process. The potassium hydroxides reacts with rapeseed oil and stearin in the mixing process to create potassium soaps. Glycerin is released.

Reaction equation (R1, R2, R3=fatty acid):

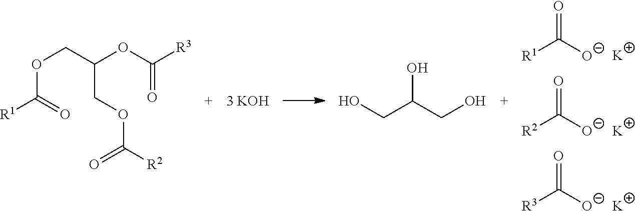

| | | | |
|---|---|---|---|
| Fatty acid ester | 3 Potassium | Glycerin | 3 Fatty acid |
| | hydroxide | | soaps |

Natural rapeseed oil is made up of various fatty acids esterified with glycerin (approx. 5%), such as oleic acid (approx. 59%), linoleic acid (approx. 21%), linolenic acid (approx. 8%), palmitic acid (approx. 6%), and stearic acid (approx. 1%). These are distinguished by chain length and number of double bonds.

Due to their reactivity, shorter-chain fatty acids saponify more easily than longer-chain fatty acids, and in the case of fatty acids having the same chain length, unsaturated fatty acids saponify more easily than saturated fatty acids (having double bonds).

Following the reaction, the composition of core mass and the crayons or cores is as follows:

| | | |
|---|---|---|
| Stearin | 15 | wt. % |
| Potassium stearate | 9.9 | wt. % |
| Potassium oleate | 8.1 | wt. % |
| Potassium linoleate | 2.8 | wt. % |
| Potassium linolenate | 1.1 | wt. % |
| Potassium palmitate | 0.9 | wt. % |
| Glycerin | 2.2 | wt. % |
| Kaolin | 55 | wt. % |
| Lithopone DS 30 | 3 | wt. % |
| Pigment 1 (Pigment red 254 5 CI 56110) | 1 | wt. % |
| Pigment 2 (Pigment red 122) | 1 | wt. % |

The core has a very soft release and also adheres to smooth surfaces due to the saponification with potassium.

### EXAMPLE 2: GREEN WATER-SOLUBLE CORE USING THE WET METHOD (SAPONIFICATION DURING MIXING)

Starting formulation:

| | | |
|---|---|---|
| Stearin | 20 | wt. % |
| Palm oil | 12 | wt. % |
| Potassium carbonate | 5 | wt. % |
| Kaolin | 58 | wt. % |
| Titanium dioxide | 2 | wt. % |
| Pigment (Pigment green 7) | 3 | wt. % |

A core or crayon or a pencil having a core with a composition as in Example 2 is produced using the wet method. An additional 20 wt. % water is added, and evaporates again at the end of the mixing process. During the mixing process, the potassium carbonate reacts with palm oil and stearin to form potassium soaps. Glycerin and carbon dioxide are released. The carbon dioxide escapes.

Reaction equation (R1. R2. R3=fatty acid):

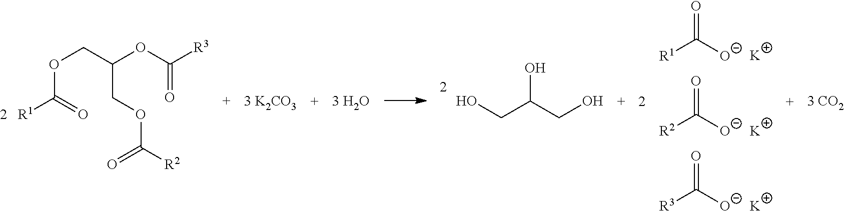

| | | | | | |
|---|---|---|---|---|---|
| 2 Fatty | 3 | 3 Water | 2 Glycerin | 6 Fatty acid | 3 Carbon |
| acid esters | Potassium | | | soaps | dioxide |
| | carbonate | | | | |

Compared to potassium hydroxide, potassium carbonate is not considered corrosive. Natural palm oil is made up of different fatty acids esterified with glycerin (approx. 4.9%), such as lauric acid (approx. 0.5%), myristic acid (approx. 0.9%), palmitic acid (approx. 41.8%), oleic acid (approx. 38.1%), linoleic acid (approx. 9.0%), and stearic acid (approx. 4.8%).

Following the reaction, the composition of the core mass and the crayons or cores is as follows:

| | | |
|---|---|---|
| Stearin | 11.3 | wt. % |
| Potassium stearate | 10.4 | wt. % |
| Potassium oleate | 5.3 | wt. % |
| Potassium palmitate | 5.8 | wt. % |
| Potassium linoleate | 1.2 | wt. % |
| Potassium myristate | 0.1 | wt. % |
| Potassium laurate | 0.1 | wt. % |
| Glycerin | 2.3 | wt. % |
| Kaolin | 58.6 | wt. % |
| Titanium dioxide | 2 | wt. % |
| Pigment (Pigment green 7) | 3 | wt. % |

The combination of fatty acid soaps ensures particularly good water solubility with good stability of the core.

### EXAMPLE 3: BLUE, WATERPROOF COLORED PENCIL CORE USING THE DRY METHOD (RAW MATERIAL ALREADY SAPONIFIED)

Since the soap is already available, in this formulation the starting formulation is the core formulation and is made up as follows:

| | | |
|---|---|---|
| Rice bran wax | 5 | wt. % |
| Sodium stearate | 30 | wt. % |
| Kaolin | 60 | wt. % |
| Pigment (Pigment blue 15:1) | 5 | wt. % |

A core or crayon or a pencil having a core with a composition as in Example 3 is produced using the dry method. The core has a soft and color-intense release, even with slight pressure or coated paper.

### EXAMPLE 4: PIGMENT-FREE BLUE COLORED PENCIL CORE WITH COLORANT USING THE WET METHOD (RAW MATERIALS ALREADY SAPONIFIED)

| | | |
|---|---|---|
| Stearin | 7 | wt. % |
| Sodium stearate | 42 | wt. % |
| Sorbitol | 7 | wt. % |
| Kaolin | 43.8 | wt. % |
| Colorant (Brilliant Blue E 133) | 0.2 | wt. % |

A core or crayon or a pencil having a core with a composition as in Example 4 is produced using the wet method. An additional 5 wt. % water is added and completely evaporates by the end of the mixing process. At the beginning the colorant is dissolved in the added water and distributed evenly in this way. The core is particularly vibrant due to the colorant and low filler content.

### EXAMPLE 5: YELLOW WATERPROOF CORE WITH HIGH PROPORTION OF WAX USING THE DRY METHOD (RAW MATERIALS ALREADY SAPONIFIED)

| | | |
|---|---|---|
| Stearin | 15 | wt. % |
| Hydrated soybean oil | 4 | wt. % |
| Microcrystalline wax | 1 | wt. % |
| Calcium stearate | 20 | wt. % |
| Sorbitol | 15 | wt. % |
| Kaolin | 40 | wt. % |
| Pigment yellow 1 | 5 | wt. % |

This core can be used for marking, since it releases softly and does not cover writing. The microcrystalline wax is contained only in a proportion of 1 wt. % and thus does not interfere with the biodegradability of the core's binder.

### EXAMPLE 6: GREEN WATERPROOF SOFT PASTEL CRAYON USING THE DRY METHOD (RAW MATERIALS ALREADY SAPONIFIED)

| | | |
|---|---|---|
| Stearin | 4 | wt. % |
| Lithium stearate | 36 | wt. % |
| Kaolin | 55 | wt. % |
| Pigment green 7 | 5 | wt. % |

Lithium stearate ensures a soft, chalky release. The application can be wiped away easily due to the low wax content.

### EXAMPLE 7: LUMINESCENT FILLER-FREE WATERPROOF COLOR CORE USING THE DRY METHOD (RAW MATERIALS ALREADY SAPONIFIED)

| | |
|---|---|
| Stearin | 10 wt. % |
| Sodium stearate | 60 wt. % |
| Sorbitol | 10 wt. % |
| Pigment (strontium aluminate) | 20 wt. % |

The filler-free formulation is semi-transparent. This makes it possible to attain a luminescent effect. The high proportion of binder makes possible tool-friendly mixing, extruding, and sharpening despite the abrasive pigment.

1. Biodegradable and/or compostable core or crayon for writing, drawing, and/or cosmetic purposes, containing at least one fatty acid ester, at least one metal soap and/or soap, and at least one colorant,
characterized in that
the at least one fatty acid ester and the at least one metal soap and/or soap are included in a total proportion of 33 to 90 wt. %.

2. Biodegradable and/or compostable core or crayon according to claim 1,
characterized in that
at least one fatty acid ester and/or at least one metal soap and/or soap based on natural fats, waxes, and/or oils are included.

3. Biodegradable and/or compostable core or crayon according to claim 2,
characterized in that
at least one fatty acid ester and/or at least one metal soap or soap based on a carboxylic acid, in particular a fatty acid such as stearic acid, palmitic acid, oleic acid, lauric acid, myristic acid, capric acid, palmtoleic acid, linoleic acid, rice bran wax, or sunflower wax, is included.

4. Biodegradable and/or compostable core or crayon according to claim 1,
characterized in that
at least one metal soap and/or soap based on metal ions of monovalent salts of alkali metals, in particular lithium, sodium, and/or potassium, and/or based on metal ions of divalent alkaline earth metals, in particular magnesium, calcium, and barium, and/or based on metal ions of metals, in particular aluminum and zinc, is included.

5. Biodegradable and/or compostable core or crayon according to claim 1,
characterized in that
a soap based on a fatty acid is included, in particular a fatty acid up to a maximum C16 and/or a mixture of longer-chain fatty acids than C18 and fatty acids to a maximum C16, and based on sodium and/or potassium as metal ion.

6. Biodegradable and/or compostable core or crayon according to claim 1,
characterized in that
a metal soap based on cations lithium, magnesium, calcium, barium, aluminum, and zinc as metal ion is included and/or based on sodium and/or potassium as metal ion is included in combination with a longer-chain fatty acid than C18.

7. Biodegradable and/or compostable core or crayon according to claim 1,
characterized in that
a mass ratio of the fatty acid(s) to metal soap(s) and/or soap(s) is between 1:1 and 1:10.

8. Biodegradable and/or compostable core or crayon according to claim 1,
characterized in that
the colorant is included in a proportion of 0.1 to 35 wt. %, in particular in a proportion of 0.1 to 25 wt. %.

9. Biodegradable and/or compostable core or crayon according to claim 1,
characterized in that
dyes and/or organic pigments and/or inorganic pigments are included as colorants, in particular organic and/or inorganic pigments having a particle size distribution D90<100 μm.

10. Biodegradable and/or compostable core or crayon according to claim 1,
characterized in that
at least one filler is included, in particular in a maximum proportion of 75 wt. % and/or wherein the at least one filler is selected in particular from the group calcium carbonate, kaolin, talc, pumice powder, quartz powder, mica, white pigments, and/or plant fibers.

11. Biodegradable and/or compostable core or crayon according to claim 1,
characterized in that
at least one additive is included, in particular in a maximum proportion of 15 wt. %, and/or wherein the additive is selected in particular from the group hydroxyls and/or carboxylic acids and/or salts.

12. Pencil having a biodegradable and/or compostable core according to claim 1, in particular with a casing made of wood or a wood substitute.

13. Method for producing a biodegradable and/or compostable core or crayon according to claim 1, characterized in that the at least one fatty acid ester, the at least one metal soap and/or soap, or at least the metal hydroxides and/or metal salts reacting to form a metal soap and/or soap, and the at least one colorant are mixed and/or homogenized and thermoplastically processed at an elevated temperature that causes melting, and the core base substance obtained therefrom is extruded to form cores or crayon strands, wherein the at least one fatty acid ester, the at least one metal soap and/or soap, or the fatty acid esters and metal hydroxides and/or metal salts reacting to form the metal soap and/or soap are added such that the at least one fatty acid and the at least one metal soap and/or soap are included in the core or crayon in a proportion of 33 to 90 wt. %.

14. Method for producing a biodegradable and/or compostable core or crayon according to claim 13, characterized in that water is added, which, together with the at least one fatty acid, the at least one metal soap and/or soap, or the fatty acids and metal hydroxides and/or metal salts reacting to form the metal soap and/or soap, and the at least one colorant, are thermoplastically processed, and wherein the proportion of water is reduced to at most 0.5 wt. %, preferably 0 wt. %, during the thermoplastic processing.

15. Method for producing a biodegradable and/or compostable core or crayon according to claim 13, characterized in that
the at least one fatty acid, the at least one metal soap and/or soap, or the metal hydroxides reacting to form metal soap and/or soap, and the at least one colorant are mixed at a temperature of at least 100° C.-150° C. and extruded at 50° C. to 140° C.
